(19) United States
(12) Patent Application Publication
(10) Pub. No.: US 2014/0180285 A1
(43) Pub. Date: Jun. 26, 2014

(54) Surgical Fasteners Including Nanomaterial Coating for Applying Pulsed Current

(71) Applicant: Covidien LP, Mansfield, MA (US)

(72) Inventor: Gregory R. Thomason, Arvada, CO (US)

(73) Assignee: Covidien LP, Mansfield, MA (US)

(21) Appl. No.: 14/102,540

(22) Filed: Dec. 11, 2013

Related U.S. Application Data
(60) Provisional application No. 61/739,867, filed on Dec. 20, 2012.

(51) Int. Cl. A61B 17/11; A61B 18/12

(52) U.S. Cl. CPC A61B 17/11; A61B 18/12
USPC 606/49; 606/153

(57) ABSTRACT

The present disclosure relates to a surgical fastener including a backspan with a first and second legs depending from each opposed end of the backspan and at least one pulse generating layer coupled to a portion of the fastener. The pulse generating layer may be coupled to a portion of the first and second legs, a portion of the backspan of the fastener, or the entire surgical fastener. The fastener may include a titanium and/or silver material. The fastener may include a first and second pulse generating layers with a dielectric portion separating the first and second pulse generating layers. The pulse generating layer may be a nanomaterial capable of embedding battery and capacitor technology to deliver a low amperage pulsed current.

### CROSS-REFERENCE TO RELATED APPLICATIONS

This application claims the benefit of and priority to U.S. Provisional Patent Application No. 61/739,867, filed Dec. 20, 2012, the entire disclosure of which is incorporated by reference herein.

### BACKGROUND

1. Technical Field

The present disclosure relates generally to surgical fasteners including a pulse generating coating for applying a pulsed current to body tissue. More particularly, the present disclosure relates to surgical fasteners for use in a surgical fastening device suitable for performing anastomosis and applying a pulsed current thereto.

2. Background of Related Art

Surgical fasteners are highly specialized fasteners that can be employed during a variety of medical procedures. Closing skin wounds, anastomosing bowels, and excising portions of a lung are just a few of the many different types of medical procedures where surgical fasteners may be employed.

Many varieties of surgical fastening apparatus are known in the art, some of which are specifically adapted for use in various surgical procedures including but not limited to, end-to-end anastomosis, circular end-to-end anastomosis, open gastrointestinal anastomosis, endoscopic gastrointestinal anastomosis, and transverse anastomosis.

Anastomosis is the surgical joining of separate hollow organ sections. Typically, an anastomosis procedure follows surgery in which a diseased or defective section of hollow tissue is removed and the remaining end sections are to be joined. Depending on the desired anastomosis procedure, the end sections may be joined by either circular, end-to-end or side-to-side organ reconstruction methods.

In an end to-end anastomosis procedure, the two ends of the organ sections are joined by means of a fastening instrument which drives a circular array of fasteners through the end section of each organ section and simultaneously cores any tissue interior of the driven circular array of fasteners to free the tubular passage. Examples of instruments for performing circular anastomosis of hollow organs are described in U.S. Pat. Nos. 6,053,390; 5,588,579; 5,119,983; 5,005,749; 4,646,745; 4,576,167; and 4,473,077, each of which is incorporated herein in its entirety by reference. Typically, these instruments include an elongated shaft having a handle portion at a proximal end to actuate the instrument and a fastener holding component disposed at a distal end. An anvil assembly including an anvil rod with attached anvil head is mounted to the distal end of the instrument adjacent the fastener holding component. Opposed end portions of tissue of the hollow organ(s) to be fastened are clamped between the anvil head and the fastener holding component. The clamped tissue is fastened by driving one or more fasteners from the fastener holding component so that the ends of the fasteners pass through the tissue and are deformed by the anvil head. The fasteners are driven from the fastener holding component by a pusher or pushers. An annular knife is concurrently advanced to core tissue with the hollow organ to free a tubular passage within the organ.

Besides anastomosis of hollow organs, surgical fastening devices for performing circular anastomosis have been used to treat internal hemorrhoids in the rectum. Typically, during use of a circular fastening device for hemorrhoid treatment, the anvil head and the fastener holding component of the surgical fastening device are inserted through the anus and into the rectum with the anvil head and the fastener holding component in an open or unapproximated position. Thereafter, a purse-string suture is used to pull the internal hemorrhoidal tissue towards the anvil rod. Next, the anvil head and the fastener holding component are approximated to clamp the hemorrhoid tissue between the anvil head and the fastener holding component. The fastening device is fired to remove the hemorrhoidal tissue and fasten the cut tissue. A surgical fastener for treating hemorrhoids is disclosed in Heinrich, U.S. Pat. No. 6,959,851, the disclosure of which is hereby incorporated by reference herein.

Various types of surgical fasteners for use with various surgical fastening devices are well known in the art, including but not limited to unitary fasteners and two-part fasteners. Unitary fasteners generally include a pair of legs adapted to penetrate tissue and connected by a backspan from which they extend. In use, subsequent to formation, some of the unitary fasteners have a “B” configuration. Typically, the two-part fastener includes legs that are barbed and connected by a backspan which are engaged and locked into a separate retainer piece that is usually located in the anvil. In use, the two-part fastener is pressed into the tissue so that the barbs penetrate the tissue and emerge from the other side where they are then locked into the retainer piece.

During each of the aforementioned surgical procedures, the tissue is initially gripped or clamped between the cartridge and anvil such that individual fasteners can be ejected from the cartridge, through the slots, and forced through the clamped tissue. Thereafter, the fasteners are formed by driving them into the depressions formed on the anvil.

Following the anastomosis, there exists a possibility of anastomotic leaks which could damage the healing process and even be fatal. Accordingly, a need exists for a means of minimizing or preventing anastomotic leaks following anastomosis procedures and improving the healing time of the two ends of tissue joined together. Additionally, a danger exists for the possibility of air leaks following pulmonary resections. Accordingly, a need exists for a means of minimizing or preventing air leaks following pulmonary resection procedures.

### SUMMARY

The present disclosure relates to a surgical fastener for use with a variety of surgical fastener applying apparatus suitable for performing a variety of surgical procedures, including but not limited to curved or circular anastomosis and/or treatment to internal walls of hollow tissue organs.

According to an aspect of the present disclosure, a surgical fastener is provided including a backspan with a first and a second leg depending from each opposed end of the backspan and at least one pulse generating layer operatively coupled to at least a portion of the surgical fastener.

The first and second legs may define a plane, and the backspan may be arcuate and project in a direction out of the plane defined by the first and second legs of the fastener. The pulse generating layer may be operatively coupled to at least a portion of at least one of the first and second legs. Additionally or alternatively, the pulse generating layer may be operatively coupled to at least a portion of the backspan of the fastener. Alternatively, the pulse generating layer may be operatively coupled to the entire surgical fastener. At least a portion of the fastener may include a titanium material and/or a silver material. The fastener may include a first pulse generating layer and a second pulse generating layer, and may further include a dielectric portion separating the first pulse generating layer and the second pulse generating layer. The pulse generating layer may be a nanomaterial capable of embedding battery and capacitor technology to deliver a low amperage pulsed current. The low amperage pulsed current may be delivered upon contacting the tissue surface.

### DESCRIPTION OF THE DRAWINGS

Various embodiments of the present disclosure are described hereinbelow with reference to the drawings, wherein:

FIG. 1 illustrates an exemplary surgical fastener applying apparatus for use with a surgical fastener in accordance with embodiments of the present disclosure;

FIG. 2 illustrates another type of surgical fastener applying apparatus that may employ the surgical fastener in accordance with the present disclosure;

FIG. 3 illustrates another type of surgical fastener applying apparatus that may employ the surgical fastener in accordance with the present disclosure;

FIG. 4 illustrates another type of surgical fastener applying apparatus that may employ the surgical fastener in accordance with the present disclosure;

FIG. 5A is a perspective view, partially shown in section, of the distal head portion of the surgical fastening device of FIG. 4 positioned within a pair of tubular tissue sections prior to fastening;

FIG. 5B is a perspective view, partially shown in section, of an anvil assembly of the distal head portion approximated against the shell assembly to approximate the pair of tubular tissue sections;

FIG. 5C is an enlarged, side cross-sectional view of a portion of the head assembly during fastening of the pair of tubular tissue sections taken along section line 5C-5C of FIG. 5B;

FIG. 5D is a cross-sectional view of the pair of tubular tissue sections after fastening by the surgical fastening device of FIG. 4;

FIG. 6A is a side perspective view of one embodiment of the surgical fastener configured for use with the surgical fastening devices of FIGS. 1-4;

FIG. 6B is a side perspective view of another embodiment of the surgical fastener configured for use with the surgical fastening devices of FIGS. 1-4;

FIG. 6C is a side perspective view of another embodiment of the surgical fastener configured for use with the surgical fastening devices of FIGS. 1-4;

FIG. 7A is a side perspective view of the surgical fastener depicted in FIG. 6A shown subsequent to formation and within adjacent tissue segments; and

FIG. 7B is a side perspective view of the surgical fastener depicted in FIG. 6B shown subsequent to formation and within adjacent tissue segments.

### DETAILED DESCRIPTION OF EMBODIMENTS

Various exemplary embodiments of the presently disclosed surgical fastener for use with various surgical fastening devices, will now be described in detail with reference to the drawings wherein similar reference characters identify similar or identical elements. In the present disclosure, it is envisioned that the fasteners disclosed herein may be utilized with various fastening instruments, such as circular fasteners, linear fasteners, transverse fasteners, including open and laparoscopic/endoscopic fasteners. In the drawings, and in the following description, the term “proximal” will refer to the end of the anvil assembly, cartridge assembly or fastening device, or component thereof, that is closest to the operator during proper use, while the term “distal” will refer to the end that is farthest from the operator, as is traditional and conventional in the art. In addition, the term “surgical fastener” should be understood to include any substantially rigid structure formed of a biocompatible material that is suitable for the intended purpose of joining together adjacent tissue portions, including but not being limited to surgical staples, clips, two-part fasteners and the like.

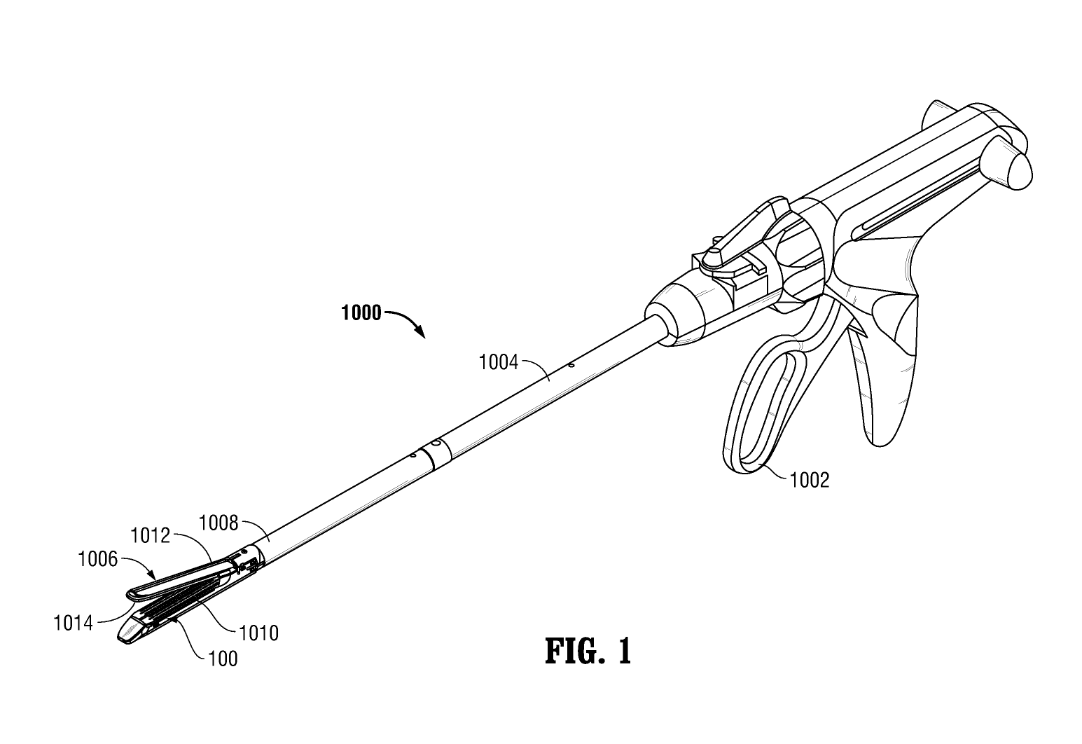

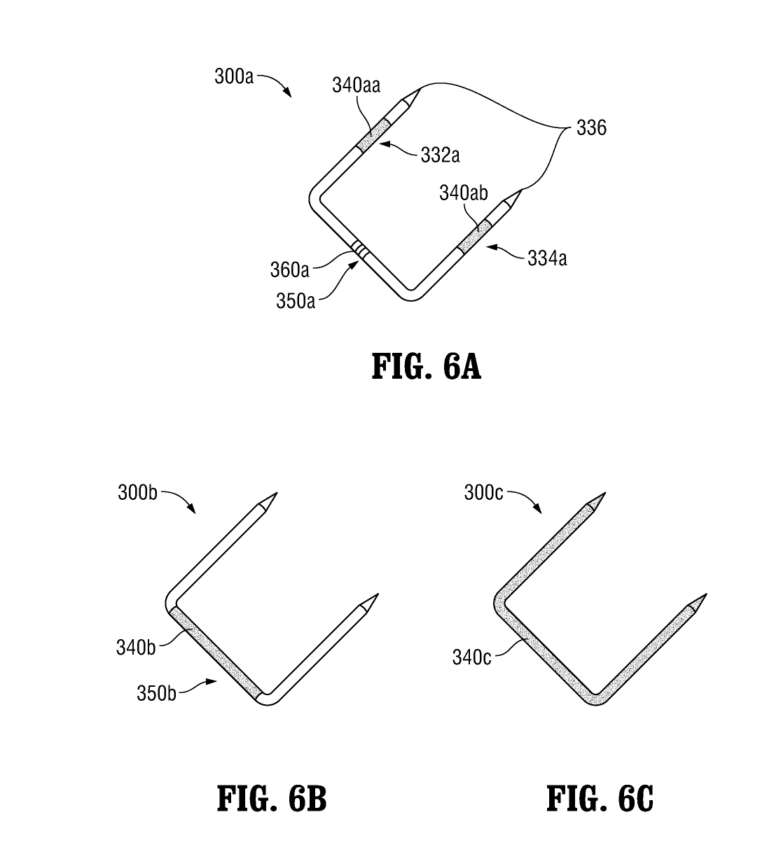

With reference to FIG. 1, a surgical fastener applying apparatus 1000 that employs a surgical fastener cartridge 100 containing the surgical fastener 300 (FIG. 6) is illustrated. Surgical fastener applying apparatus 1000 is used to sequentially apply a plurality of surgical fasteners 300 (FIG. 6) to a patient's tissue. Surgical fastener apparatus 1000 may be configured for use, subsequent sterilization, and reuse, or may be configured for single use. Surgical fastener applying apparatus 1000 includes a handle 1002, an elongated shaft or endoscopic portion 1004 extending distally therefrom, and an operative tool 1006 coupled to a distal end 1008 of the elongated shaft 1004. In general, operative tool 1006 is adapted to clamp, sequentially fasten together, and sever adjacent tissue segments along a cut-line. Operative tool 1006 includes a pair of opposed jaws 1012, 1010 pivotally coupled to one another and respectively including an anvil member 1014 that is approximated relative to cartridge 100 during use. The anvil includes depressions that are aligned with, and/or are in registration with slots defined in the cartridge 100, through which the fasteners 300 (FIG. 6) will emerge, to effectuate formation. For a more detailed discussion of the approximation and firing of surgical fastener applying apparatus 1000, reference is made to commonly owned U.S. Pat. No. 5,865,361, currently assigned to Tyco Healthcare Group LP, the entire contents of which is incorporated herein by reference. In some embodiments, the cartridge and/or anvil is removable and replaceable.

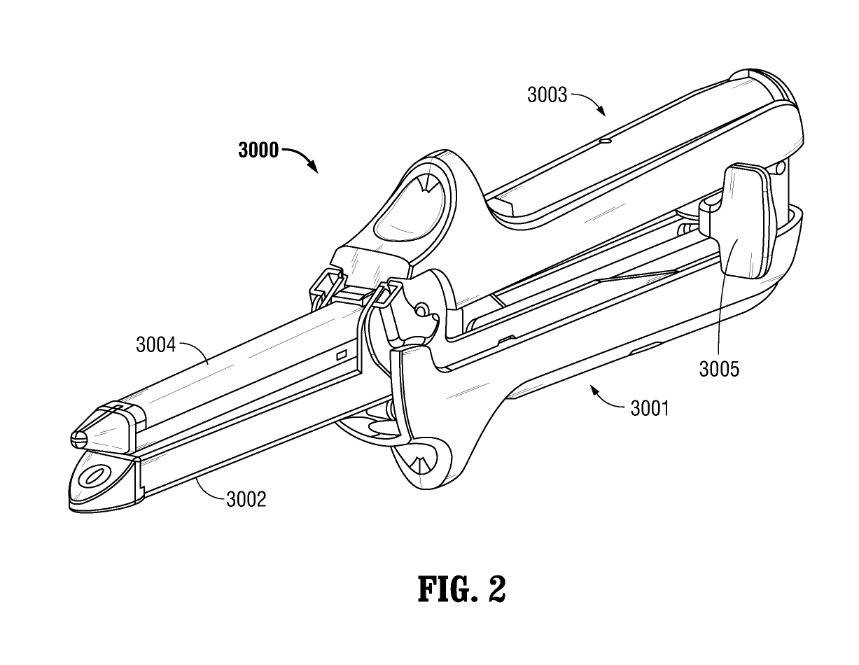

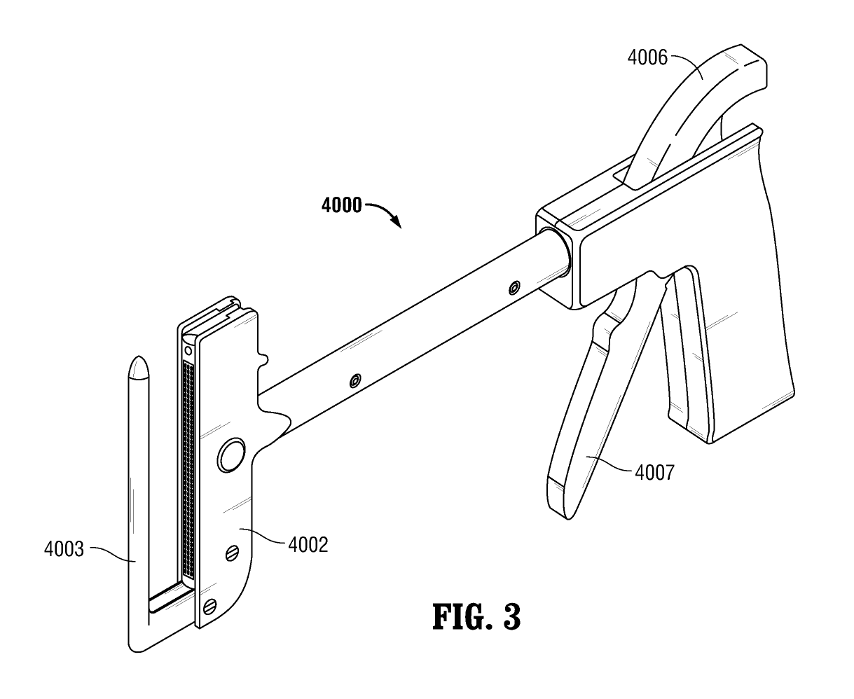

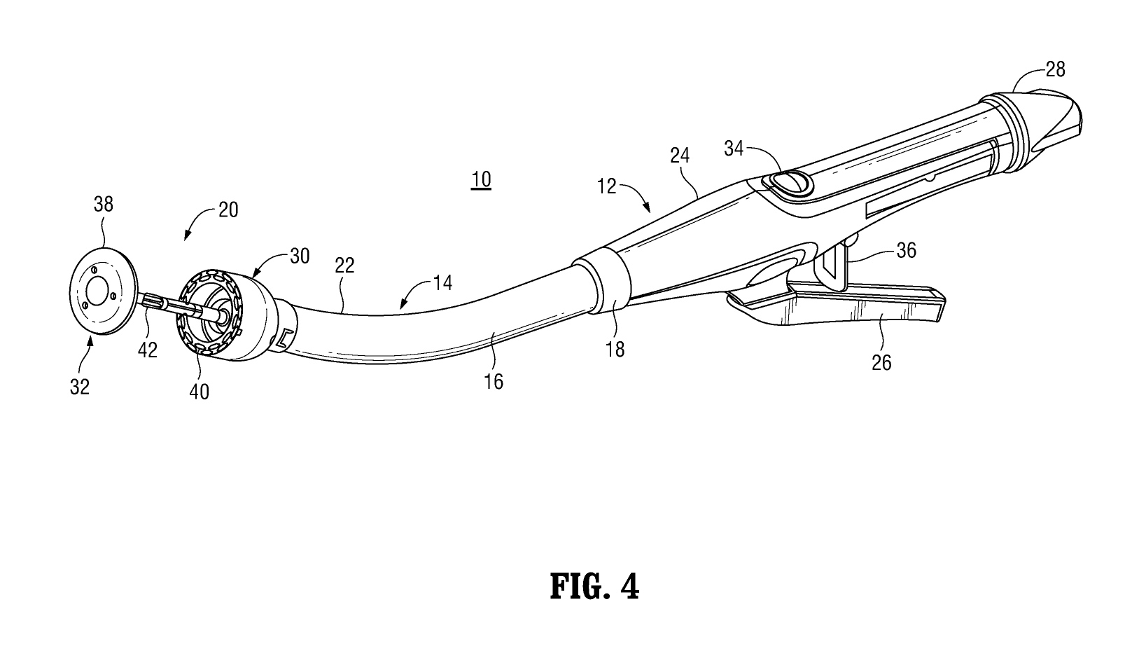

While surgical fastener applying apparatus 1000 is depicted as an apparatus suitable for use in laparoscopic procedures for performing surgical anastomotic fastening of tissue, those skilled in the art will appreciate that cartridge 100, and/or the fasteners 300 (FIG. 6) contained therein, may be adapted for use with any surgical instrument suitable for the intended purposes described herein. For example, cartridge 100 may be adapted for use with an end-to-end anastomosis device 10, as seen in FIG. 4, wherein the fasteners 300 (FIG. 6) are arranged in substantially annular rows; and/or a surgical stapling instrument 3000, as seen in FIG. 2, for use during an open gastro-intestinal anastomotic stapling procedure wherein the fasteners 300 (FIG. 6) are arranged in substantially linear rows; and/or a surgical fastening instrument 4000, as seen in FIG. 3, wherein the fasteners 300 (FIG. 6) are arranged in substantially linear rows, or, for example, any of the surgical fastener applying apparatus disclosed in U.S. Pat. Nos. 6,045,560; 5,964,394; 5,894,979; 5,878,937; 5,915,616; 5,836,503; 5,865,361; 5,862,972; 5,817,109; 5,797,538; and 5,782,396, which are each incorporated by reference herein in their entirety. The cartridge 100 in preferred embodiments may be removable and replaceable with another loaded cartridge 100 for use with these apparatus.

Turning specifically to FIG. 4, there is illustrated an embodiment of the presently disclosed surgical fastening device 10. Surgical fastening device 10 generally includes a proximal handle assembly 12, an elongated central body portion 14 having an elongated outer tube 16 extending distally from a distal end 18 of handle assembly 12, and a distal head portion 20 mounted on a distal end 22 of elongated outer tube 16. Handle assembly 12 includes a stationary trigger 24 and a firing trigger 26 movably mounted on stationary trigger 24 and a rotatable approximation knob 28. Surgical fastening device 10 includes internal components and functions substantially similar to that disclosed in U.S. Pat. No. 7,857,187, the entire disclosure of which is incorporated by reference herein.

Distal head portion 20 includes a shell assembly 30 mounted on distal end 22 of elongated outer tube 16 and an anvil assembly 32 movable relative to shell assembly 30 to capture tissue sections to be fastened therebetween. Anvil assembly 23 is movable, in response to rotatable approximation knob 28, between an open position, spaced from shell assembly 30, to a closed position, substantially adjacent shell assembly 30 for fastening tissue. An indicator 34 is provided on stationary handle 24 to give the user a visual indication of the position of anvil assembly 32 relative to shell assembly 30 to ensure tissue is properly clamped prior to fastening. A lock lever 36 is pivotally mounted on stationary handle 24 and is engagable with firing trigger 26 to prevent premature firing of surgical fastening device 10.

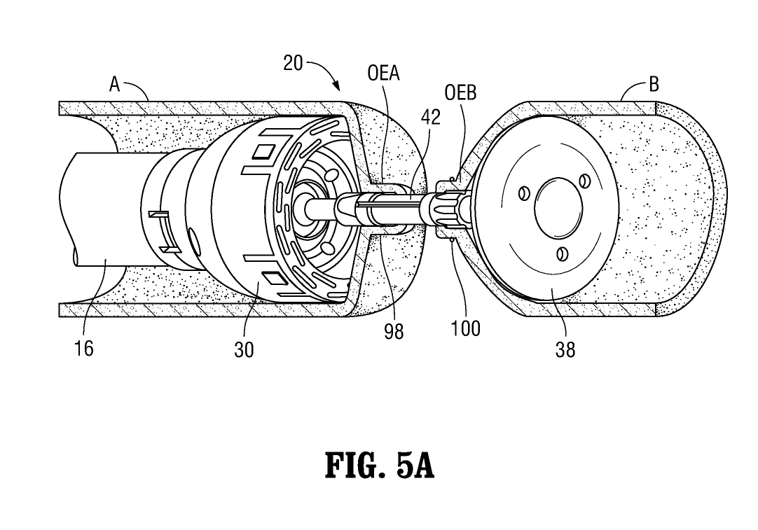

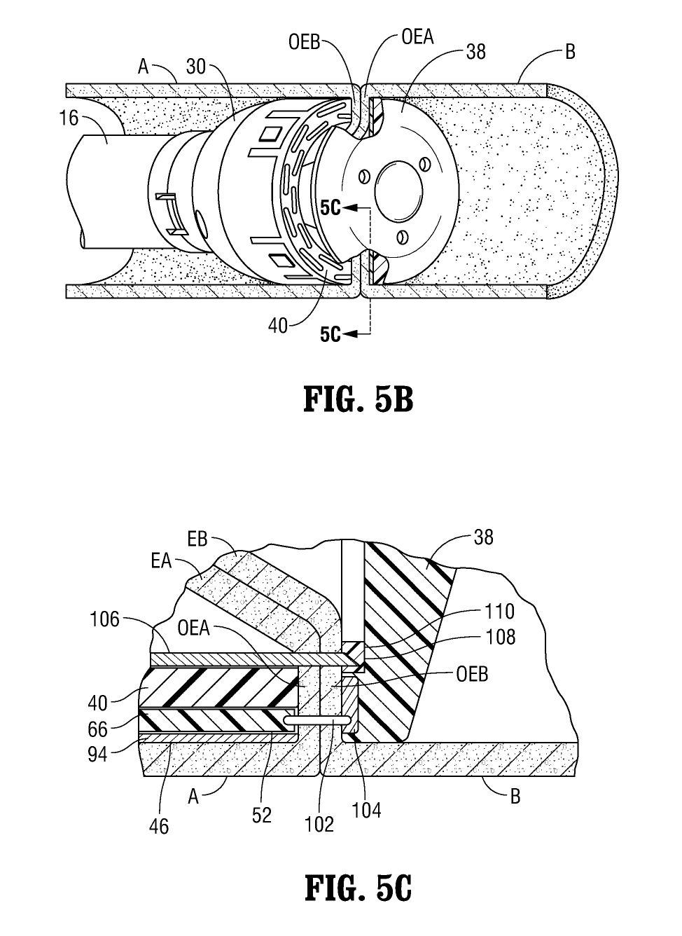

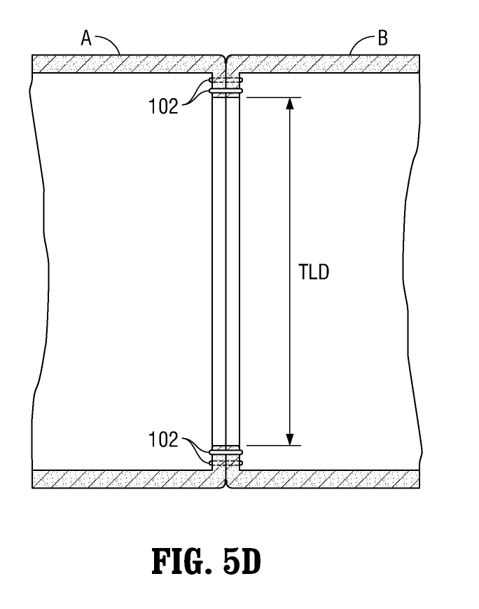

Turning now to FIGS. 5A-5D, and with continued reference to FIG. 4, the use of surgical fastening device 10 to reconnect a pair of tubular tissue sections A and B will now be described. Referring initially to FIGS. 4 and 5A, fastener clinching anvil member 38 is initially an extended position relative to shell assembly 30. Distal head portion 20 of surgical fastening device 10 is inserted into a pair of tubular tissue sections A and B such that fastener clinching anvil member 38 resides within tubular tissue section B and shell assembly 30 resides within tubular tissue section A. As best shown in FIG. 5A, an open end OEA of tubular tissue section A is secured against anvil retainer 42 by a purse string suture 98 and an open end OEB of tubular tissue section B is secured against anvil retainer 42 by a purse string suture 100.

Referring to FIGS. 4, 5A, and 5B, rotatable approximation knob 23 on handle assembly 12 is rotated to draw anvil retainer 42, and thus fastener clinching anvil member 38, proximally toward shell assembly 30. This clamps open end OEA of tubular tissue section A firmly against open end OEB of tubular tissue section B between fastener clinching anvil member 38 and shell assembly 30. Once the user has received a visual indication via indicator 34 on handle assembly 12 that tissue sections A and B have been properly clamped by distal head portion 20, lock lever 36 is released and firing trigger 26 is compressed against stationary handle 24 to actuate surgical fastening device 10.

With specific reference to FIG. 5C, actuation of surgical fastening device 10 drives extending fingers 66 distally through a fastener pockets 52 in fastener guide 40. Fasteners 102, are driven out of fastener pockets 52, through open end OEA of tubular tissue section A and open end OEB of tubular tissue section B and into fastener clinching pockets 104 formed in anvil member 38. This securely fastens open end OEA of tubular tissue section A to open end OEB of tubular tissue section B. Thereafter, a knife blade 106 is driven through tubular tissue sections A and B and into a backing ring 108 formed in anvil member 38 causing a sharp distal tip 110 of knife blade 106 to sever excess tissue EA and EB located radially inwardly of fasteners 102.

Turning now to FIGS. 6A-6C, fasteners 300a, 300b, and 300c will now be described and referred to collectively as fasteners 300 throughout this description. Fasteners 300 are substantially similar to fasteners 102 described above, and will only be described to the extent that they differ. Fasteners 300 may be formed of a stainless steel wire of circular cross-section, although other materials and wire shapes are contemplated. Additionally, fasteners may be formed entirely or partially of silver, titanium, and/or any other conductive or non-conductive material. The present disclosure contemplates that surgical fastener 300 may also be configured as a directionally biased fastener, such as those described in commonly owned U.S. Pat. No. 7,398,907, the entire contents of which are incorporated by reference herein.

As described above, fastener 300 may have a circular cross-section throughout its length. It is envisioned that fastener 300 may have a variety of different cross-sections including rectangular, oval, square, triangular, trapezoidal, etc. It is also envisioned that backspan 350 and legs 332 and 334 may have different cross-sectional shapes, e.g., backspan 350 may have a rectangular cross-section and legs 332 and 334 may have an oval cross-section. Legs 332 and 334 may diverge slightly, although other configurations are envisioned, i.e., legs 332 and 334 may be substantially parallel, converge, etc.

With particular reference to FIG. 6A, fastener 300a is shown with a pair of legs 332a, 334a which are joined by a backspan 350a. Legs 332a, 334a may extend from backspan 350a such that they are substantially parallel. Alternatively, legs 332a, 334a may converge or diverge from backspan 350a, as described above.

Each of the legs 332a, 334a terminates in a penetrating end 336 that is configured to penetrate tissue and/or other suitable material (blocking and/or retainer material for example). The penetrating ends 336 of legs 332a, 332b may be tapered to facilitate the penetration of tissue, or alternatively, the penetrating ends 336 may not include a taper. In various embodiments, penetrating ends 336 may define a conical or flat surface. In some embodiments, one or both of legs 332a, 334a may be barbed as described above. Having legs 332a, 332b configured in such a manner may facilitate maintaining the surgical fastener 330 in a fixed position within the tissue and/or blocking material.

Continuing with reference to FIG. 6A, leg 332a of fastener 300a is shown with a first pulse generating layer 340aa and leg 334a of fastener 300a is shown with a second pulse generating layer 340ab. Additionally, backspan 350a of fastener 300a is shown as separated by a dielectric portion 360a formed within backspan 350a. Dielectric portion 360a may serve to electrically separate leg 332a from leg 334a, thus separating the direct conductivity between first pulse generating layer 340aa and second pulse generating layer 340ab.

First and second pulse generating layers 340aa, 340bb may include a nanomaterial capable of embedding battery and/or capacitor technology in a carbon-based substance to generate a pulsed current directly to a surgical wound to enhance healing. By applying the carbon nanomaterial with energy storage and delivery capacity to each fastener 300 a low amperage current may be introduced to the surgical site which may aid and encourage the anastomotic healing process.

Turning now to FIG. 6B, fastener 300b is shown which is substantially similar to fastener 300a. Therefore, only the differences between the two will be discussed. Fastener 300b may include a pulse generating layer 340b defined on backspan 350b. Pulse generating layer 340b may be defined on the entire backspan 350b or a portion of the backspan 350b.

Turning now to FIG. 6C, fastener 300c is shown which is substantially similar to fasteners 300a and 300b. Therefore, only the differences will be discussed below. Fastener 300c is shown with pulse generating layer 340c defined on the entire fastener 300c. Although fastener 300c is shown as being entirely layered by pulse generating layer 340c, it is also appreciated that all or a portion of fastener 300c may be layered by pulse generating layer 340c.

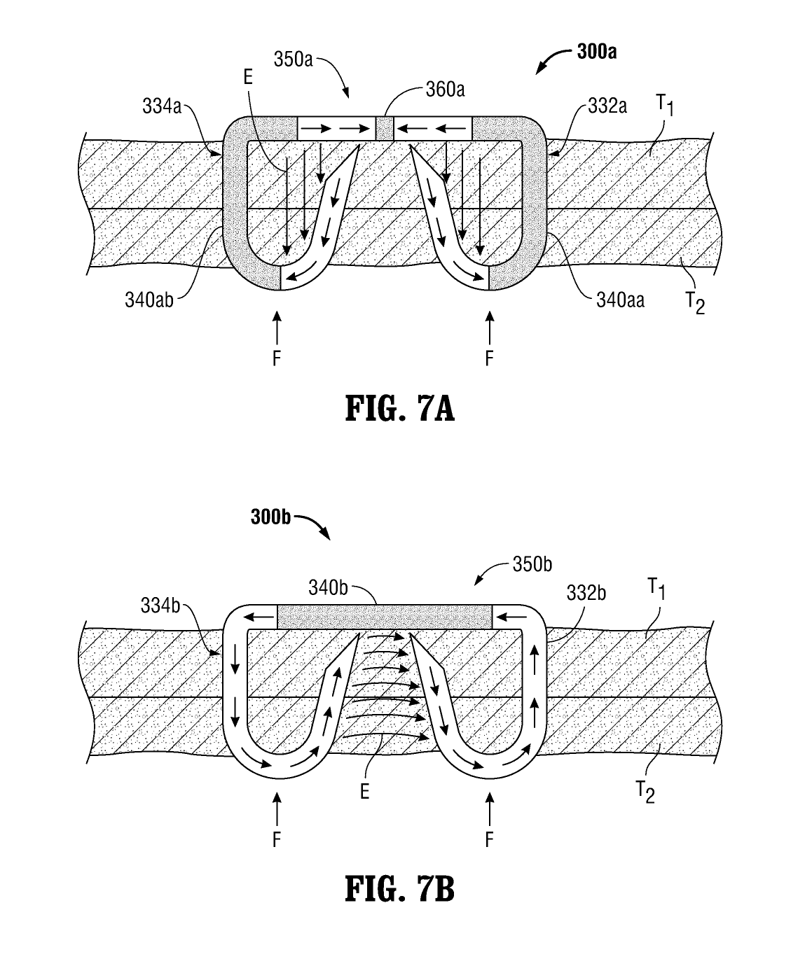

Turning now to FIG. 7A, surgical fastener 300a is shown subsequent to formation. Surgical fastener 300a is configured to provide a compression force to fastened tissue occupied therein. To this end, legs 332a, 334a cooperate with backspan 350a to maintain adjacent tissue segments “T1,” “T2” in approximation and apply a compressive force “F” thereto. The compressive force “F” applies pressure to the tissue segments “T1,” “T2”, thereby restricting the flow of blood through the tissue surrounding the surgical fastener 300a and facilitating hemostasis. The configuration of the backspan 350a may limit the amount of pressure that can be applied to the tissue segments “T1,” “T2” such that the flow of blood through the tissue is not completely restricted. When formed, the surgical fastener 300a has a generally “B” shape with an overall height measured from the backspan 350a to the outermost curve of the legs 332a, 334a.

Continuing with reference to FIG. 7A, upon formation of surgical fastener 300a, or upon contact of pulse generating layers 340aa and 340ab to tissue segments “T1” and “T2,” pulse generating layers 340aa and 340ab deliver a pulsed current directly to tissue segments “T1” and “T2.” For example and without limitation, electrons “E” are shown traveling from tissue segment “T1” to tissue segment “T2” to enhance healing. Pulsed current may be a low amperage current which encourages the healing process of the two segments of tissue “T1” and “T2.”

Turning now to FIG. 7B, surgical fastener 300b is shown subsequent to formation. Surgical fastener 300b is configured to provide a compression force to fastened tissue occupied therein. To this end, legs 332b, 334b cooperate with backspan 350b to maintain adjacent tissue segments “T1,” “T2” in approximation and apply a compressive force “F” thereto. The compressive force “F” applies pressure to the tissue segments “T1,” “T2”, thereby restricting the flow of blood through the tissue surrounding the surgical fastener 300b and facilitating hemostasis. The configuration of the backspan 350b may limit the amount of pressure that can be applied to the tissue segments “T1,” “T2” such that the flow of blood through the tissue is not completely restricted. When formed, the surgical fastener 300b has a generally “B” shape with an overall height measured from the backspan 350b to the outermost curve of the legs 332b, 334b.

Continuing with reference to FIG. 7B, upon formation of surgical fastener 300b, or upon contact of pulse generating layer 340b to tissue segment “T1,” pulse generating layer 340b delivers a pulsed current directly to tissue segment “T1.” Additionally, or alternatively, electrons E may flow in either direction from leg 334b, through tissue segments “T1” and “T2,” to leg 332b as shown by arrows E.

For the surgical fastener, end effector, and/or anvil assembly discussed above, two annular rows of surgical fasteners, two annular rows of fastener retention slots and two annular rows of fastener forming pockets may be used. The annular rows can be circular, ovoid, semi-circular, or in the shape of an arc. Additionally, a longitudinal axis of each fastener retention slot and fastener forming pocket may be angled or bent in order to approximate an arc. The rows of fastener retention slots and rows of fastener forming pockets with curved lengths can be used in surgical fasteners and end effectors that have linear jaws or jaws of other shapes.

While the above description contains many specifics, these specifics should not be construed as limitations on the scope of the present disclosure, but merely as illustrations of various embodiments thereof. Therefore, the above description should not be construed as limiting, but merely as exemplifications of various embodiments. Those skilled in the art will envision other modifications within the scope and spirit of the disclosure.

What is claimed:

1. A surgical fastener, comprising:
a backspan with a first and second leg depending from each opposed end of the backspan; and
at least one pulse generating layer operatively coupled to at least a portion of the surgical fastener.

2. The surgical fastener according to claim 1, wherein the first and second legs define a plane, and wherein the backspan is arcuate and projects in a direction out of the plane defined by the first and second legs of the surgical fastener.

3. The surgical fastener according to claim 1, wherein the pulse generating layer is operatively coupled to at least a portion of at least one of the first and second legs.

4. The surgical fastener according to claim 1, wherein the pulse generating layer is operatively coupled to at least a portion of the backspan of the surgical fastener.

5. The surgical fastener according to claim 1, wherein the pulse generating layer is operatively coupled to the entire surgical fastener.

6. The surgical fastener according to claim 1, wherein at least a portion of the fastener includes a titanium material.

7. The surgical fastener according to claim 1, wherein at least a portion of the fastener includes a silver material.

8. The surgical fastener according to claim 1, wherein the surgical fastener includes a first pulse generating layer and a second pulse generating layer, and further includes a dielectric portion separating the first pulse generating layer from the second pulse generating layer.

9. The surgical fastener according to claim 1, wherein the pulse generating layer is a nanomaterial capable of embedding battery and capacitor technology to deliver a low amperage pulsed current.

10. The surgical fastener according to claim 9, wherein the low amperage pulsed current is delivered upon contacting a tissue surface.
